(19) United States
(12) Patent Application Publication
(10) Pub. No.: US 2009/0287122 A1
(43) Pub. Date: Nov. 19, 2009

(54) LIMB PROTECTION SYSTEM

(75) Inventor: Andrew James Evans, Pymble (AU)

Correspondence Address: KNOBBE MARTENS OLSON & BEAR LLP, 2040 MAIN STREET, FOURTEENTH FLOOR, IRVINE, CA, 92614, US

(73) Assignee: HYDROSKIN PTY LTD., Baulkham Hills (AU)

(21) Appl. No.: 12/338,888

(22) Filed: Dec. 18, 2008

Related U.S. Application Data
(63) Continuation-in-part of application No. 10/563,547, filed on Jun. 12, 2006, now abandoned.

(30) Foreign Application Priority Data
Jul. 4, 2003 (AU) 2003903424

(51) Int. Cl. A61F 5/00

(52) U.S. Cl. 602/3

(57) ABSTRACT

A limb protection device and a method for applying the limb protection device to a user. The device comprises: an enclosure for enclosing the limb section; and at least one flexible cuff integral with and away from which the enclosure extends, the cuff defining an opening into the device and having a tapered profile arranged to allow, in use, the selection of an appropriately sized opening into the device, whereby the appropriately sized opening in the cuff is selected to stretch around and compress against that part of the user's limb extending therethrough in use, to prevent the ingress of matter into or egress of matter out of the device immediately at the device opening. The device may protect a section of a user's limb.

### CROSS-REFERENCE TO RELATED APPLICATIONS

This application is a continuation-in-part application of U.S. patent application Ser. No. 10/563,547, having a §371(c) date of Jun. 12, 2006, titled “LIMB PROTECTION SYSTEM,” which is a national phase of International Application No. PCT/AU2004/000903, filed Jul. 5, 2004, which claims the benefit of Australian Patent Application No. 2003903424, filed Jul. 4, 2003. Each of the foregoing applications is incorporated by reference in its entirety and made part of this specification.

### BACKGROUND

1. Field

The present invention relates to improvements in a limb protection system for protecting a section of a user's limb against the ingress of moisture, fluids such as water, and unintentional contact such as “bumping”. The invention will be described primarily with reference to its use in medical/treatment applications, but it should be appreciated that the invention can be employed wherever it is necessary or desirable to protect a section of a user's limb against moisture, contact or the like. In addition, when the term “limb” is used herein, it is not simply intended to be limited to legs and arms of a user, but conceivably can extend to the torso etc of the user as appropriate, and not just human users, but animal users.

2. Description of the Related Technology

In medical treatment, it is often necessary/important to isolate a certain section of a limb from the remainder of the body of the user, to prevent it from contacting certain liquids (especially water), solvents, surfaces, etc. For example, where the skin of a user's limb has been cut, grazed, burnt, bandaged, plastered, etc., it is often necessary/desirable to prevent that area from being subjected to contact with water, other liquids or even gases.

The applicant has previously filed a PCT application no. WO 02/24014 entitled “Limb Protection System”. The reader is referred to this application.

### SUMMARY

In a first aspect, the present invention provides a limb protection device for protecting a section of a user's limb, the device comprising:
an enclosure for enclosing the limb section; and
at least one flexible cuff integral with and away from which the enclosure extends, the cuff defining an opening into the device and having a tapered profile arranged to allow, in use, the selection of an appropriately sized opening into the device, whereby the appropriately sized opening in the cuff is selected to stretch around and compress against that part of the user's limb extending therethrough in use, to prevent the ingress of matter into the device immediately at the device opening.

In the prior art, the applicant has disclosed a device that enclosed a limb to prevent matter, such as moisture (especially water) from reaching the limb section. This is highly advantageous in many medical situations (eg. in bathing and showering of patients with wounds, bandages, plaster etc). The invention of the prior art made use of a tight fitting cuff to provide a stronger seal at the user's limb. However, as different users have different sized limbs, there was a need to manufacture the device in a variety of different sizes, each having a different sized cuff. Correspondingly, hospitals and doctor's surgeries were required to stock the device in a variety of sizes, in order to be able to apply the device to a wide range of users. The present invention preferably provides a “one size fits all” device, as the user may simply cut or tear off the cuff at an appropriate point along the tapered cuff to produce a device which provides a cuff which is sized to fit correctly and tightly around the limb of a user.

The cuff may further comprise a series of spaced-apart protrusions extending along the length of the cuff, wherein the protrusions provide structural integrity to the cuff.

The protrusions may be integrally moulded into the cuff.

The protrusions may be of a substantially semi-circular shape.

The cuff may have a width greater than a wall thickness of the enclosure.

In one embodiment, the cuff may be a continuation of the enclosure and may be formed from the same material as the enclosure, optionally having the same thickness as the enclosure.

In an alternative embodiment, the cuff may be formed from a different material, or a more dense form of the same material as the enclosure. In this regard, the cuff may be formed from a resilient polymetric material such as latex, elastic impregnated plastic, etc. to enable its stretching during fitting and removal of the device to a limb.

The enclosure may be a sleeve that is either:
Closed at one end, with the cuff located at an opening at the other opposing end; or
Open at opposing ends, with respective cuffs located at each end.

In an embodiment with the sleeve in accordance with arrangement (a), the device may be used isolating entire feet, hands, legs or arms, whereas arrangement (b) may be used for isolating particular sections of limbs (e.g. a forearm, wrist, shin or thigh) or for isolating joints (such as the elbow, shoulder, knee or hip).

In an alternative embodiment the sleeve in (a) may be releasably openable at one end for enabling access to the limb section when the device is fitted to the user (e.g. for medical access, or to let air in, or for scratching, adjustments etc.) In this regard, a press-seal opening may be employed at said one end.

The sleeve in (a) may be adapted for receiving a user's hand or foot therein.

The sleeve in (b) may be adapted for receiving the limb right therethrough with the cuff being located on opposing sides of the limb section that is enclosed within the enclosure.

The enclosure and/or the cuff may be transparent, and may be formed from a polymer.

A therapeutic agent may be provided in the interior of the device to aid in the treatment of medical conditions.

### BRIEF DESCRIPTION OF THE DRAWINGS

Notwithstanding any other forms which may fall within the scope of the present invention, preferred forms of the invention will now be described, by way of example only, with reference to the accompanying drawings in which:

FIG. 1 shows a side elevation of a first embodiment of the limb protection device in accordance with the present invention;

FIG. 2 shows a side elevation of a second embodiment of the limb protection device in accordance with the present invention.

FIG. 3 shows in cross sectional side elevation an embodiment of a device, when in use, in accordance with the present invention;

FIG. 4 shows a side elevation of a further alternative device to those shown in FIGS. 1 to 3;

FIGS. 5 and 6 show, in side elevation, two further alternative limb protection devices for use with a foot/lower leg of a user;

FIGS. 7 and 8 show, in side elevation, two further alternative devices for use with a user's leg;

FIG. 9 shows, in a side elevation, another limb protection device in accordance with the present invention; and

FIG. 10 shows, in side elevation, yet a further limb protection device in accordance with the present invention.

### DETAILED DESCRIPTION OF EMBODIMENTS

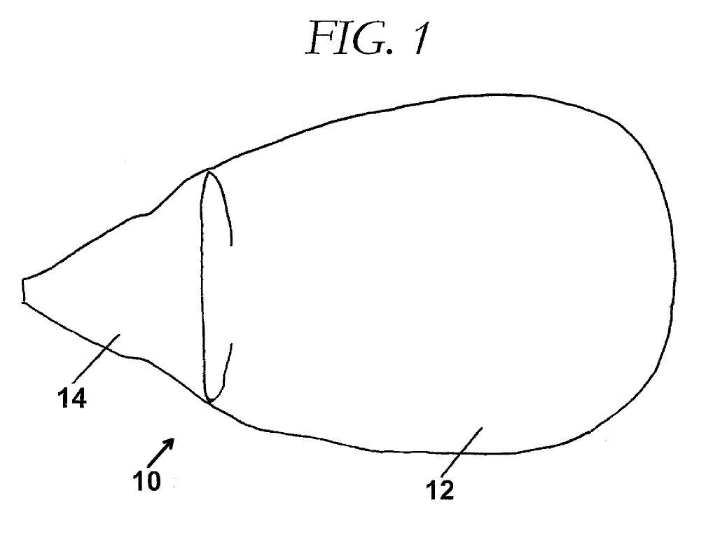

Referring to the drawings, and in particular to FIG. 1, a limb protection device in the form of a protective bag 10 includes a sleeve portion 12 and an integral cuff portion 14. The integral cuff portion has a tapered profile as shown in FIG. 1. The cuff may also, in another embodiment, taper to a “point”. Typically both the sleeve portion and cuff portion are formed from thermoplastic polymers such that they can be moulded integrally together during original formation of the protective bag.

Alternatively, the cuff portion can be formed from a stretchable, resilient polymer, such as a latex or an elastic impregnated polymer to provide a close fit around the user's limb.

Typically the cuff portion tapers from a diameter which is less than the limb of a “small” user, to a diameter which is greater than the limb of a “large” user. This allows the cuff to be sized by allowing the user (or another individual) to cut off or remove a portion of the cuff so that the resultant cuff clasps peripherally around the limb when fitted. It will be understood that the cuff may be sized by an end user, or it may be sized at the point of manufacture. The resultant cuff, once sized, provides a seal to maximize the sealing of the limb against the ingress of matter (especially moisture). It has been observed that not only can the cuff seal against the ingress of any type of liquid, but it can also deal out gas, and lock air into the bag. Thus, the bag can also function like a balloon to protect the section of limb enclosed therein against inadvertent contact, bumping, etc. The seal also prevents the egress of matter from the bag. Therefore, the bag may be filled with ice, with an anti-microbial agent, with a burn treatment cream, or with any other suitable fluid, solid or other therapeutic agent to treat the enclosed limb. The inclusion of the therapeutic agent may preferably allow for more speedy recovery of cuts, bruises, infections, burns, or other ailments.

Furthermore, in one embodiment, the device may be manufactured in sterile conditions and both ends may be sealed, such that the interior of the device remains in a sterile condition until use. If both ends are sealed at manufacture, the interior surface of the device may also be pre-coated with a suitable therapeutic agent prior to sealing.

The embodiment described herein provides a number of advantages, particularly in situations where quick isolation of a injured limb will greatly accelerate the healing process.

For example, when a burn victim is rescued from a fire, the paramedic treating the burn victim may cut an appropriately sized cuff from a device in accordance with an embodiment of the present invention, and immediately apply the device to the patient. This may provide instant relief to the burn victim, while preventing the burn site from becoming infected.

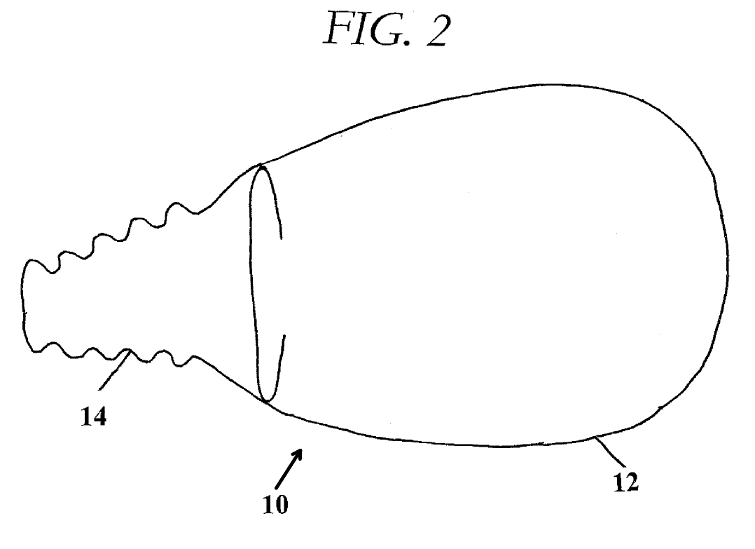

In an alternate embodiment, shown in FIG. 2, the tapered cuff preferably includes a number of spaced apart protrusions or “ribs”. The ribs serve a dual purpose.

Firstly, the ribs provide further strength to the cuff portion, particularly whilst the user is stretching the cuff to insert a limb into the device. That is, the ribs prevent the cuff from breaking or splitting while the cuff is in a stretched position (i.e. as it is being fitted to the user). As a corollary, the extra strength provided by the ribs allow the cuff to be formed of a thinner material than the material which would normally be used in forming a device with a straight tapered profile. This preferably allows the device to be manufactured at a lower cost.

Secondly, the ribs serve as a visual guide, allowing the user to more easily perceive and select the correct region in which to cut or tear off a portion of the cuff, when selecting an appropriate size for the cuff.

Generally, a user would use a knife or a pair of scissors to select an appropriate length for the cuff. However, it will be understood that the cuff may also incorporate means for assisting the user in selecting the appropriate cuff size. For example, the cuff may include, at defined intervals, a series of “weakened zones”, which allow a user to tear away a portion of the cuff without the need for cutting implements such as knives or scissors.

The cuff may alternatively or additionally have pre-printed cut-lines to indicate appropriate cutting locations for a given user's limb size.

Typically the sleeve portion 12 is formed from a transparent polymer (such as polyethylene, polypropylene etc.) to enable the user (and medical personnel) to maintain observation of the limb section enclosed therein.

The sleeve portion 12 and/or the cuff portion 14 may be coloured any one of a number of appropriate colours. The colouring may be used to indicate a particular property or feature of the device. For example, one colour (such as blue) may indicate that the device was manufactured under sterile conditions. Another colour, such as red, may indicate that a device is not manufactured under sterile conditions. Similarly, where the device includes therapeutic agent (i.e. the inside of the device is pre-coated with an appropriate therapeutic agent, such as an anti-microbial agent, a burn treatment cream, etc.), other colours may be used to denote or easily identify different devices. For example, a device containing an anti-microbial agent may be coloured yellow, whereas a device containing a burn treatment cream may be coloured green. Any appropriate colour may be used to allow users to easily distinguish between different types of devices.

Furthermore, where the device is manufactured in a number of different sizes, the different sizes may also be denoted by a different colour or different pattern applied to the surface of the device.

In embodiments which envisage the provision of two cuffs (for example, where a device is to be placed over a limb but is to leave a hand or foot exposed), the device may include a different colouring at each end of the device. This may allow a user to easily identify which cuff should be cut to a particular size (as each cuff may need to be differently sized, depending on the anatomy of the user). It will be understood that in place of a particular colour, a particular pattern or some other type of marker may be used to differentiate between different cuffs on the device. Such variations are within the purview of a person skilled in the art.

The following paragraphs provide examples of an embodiment of the device in use on a patient (i.e. after the cuff has been appropriately sized).

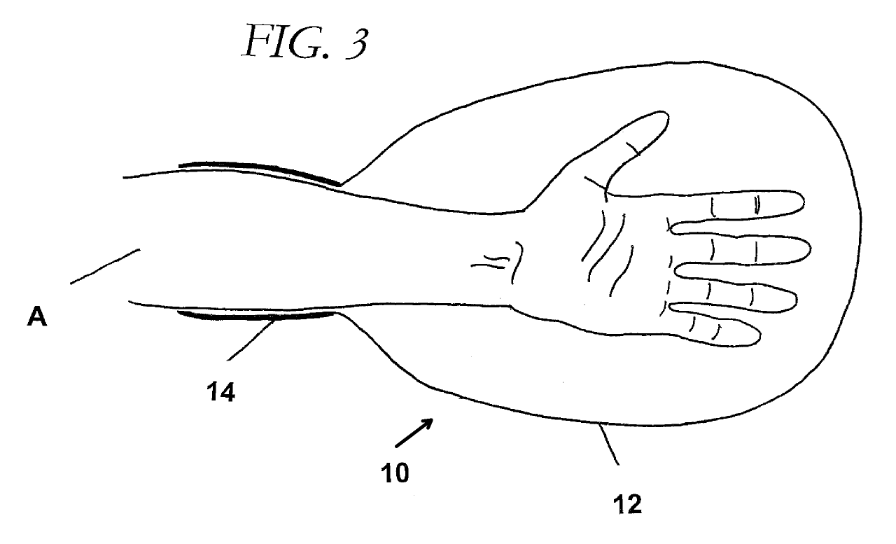

Referring now to FIG. 3, there is shown a bag typically adapted for enclosing the hand/wrist of a user. The cuff portion 14 has been cut to an appropriate size. The cuff portion 14 has been cut to an appropriate size. The cuff portion 14 is defined simply by a continuation of the sleeve portion 12 (i.e. is formed of the same material as the sleeve portion). Optionally, the cuff portion may have a thicker wall, or may be formed from a higher density version of the same material (e.g. a low density polyethylene sleeve portion and a high density polyethylene portion). Further, the cuff portion in FIG. 3 can be impregnated by an elastic material.

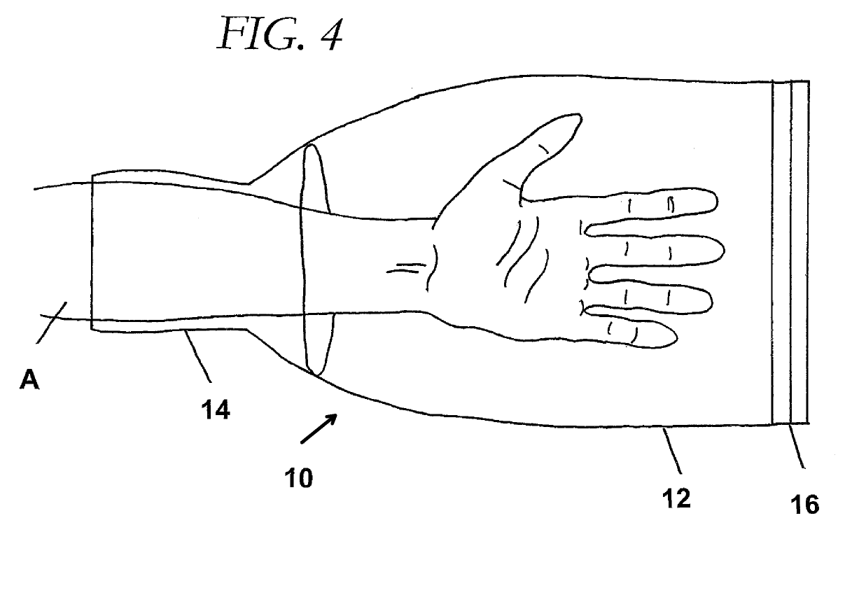

Referring now to FIG. 4, the end of the bag opposing the cuff portion may be resealably openable, for example by having a press-seal opening 16 formed thereat. Other types of resealable openings can also be employed, such as those employing resealable waterproof contact adhesives, etc.

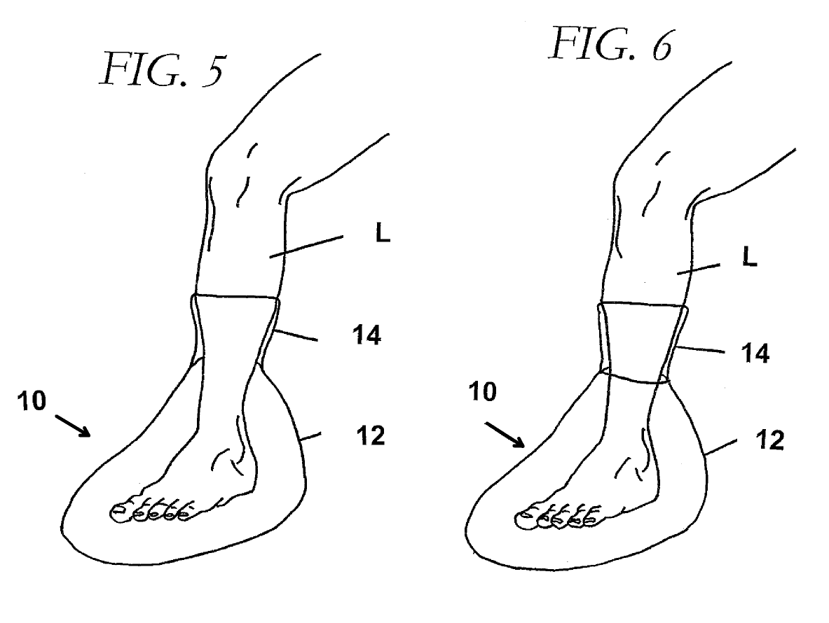

Referring now to FIGS. 5 and 6 where like reference numerals are used to denote similar or like parts, the cuff portion 14 surrounds a shin and calf of the user to enclose the foot/ankle of the user. Either the same protective bag can be used for both the hand/wrist and the foot/ankle, or the bag can be a different size for foot/ankle usage.

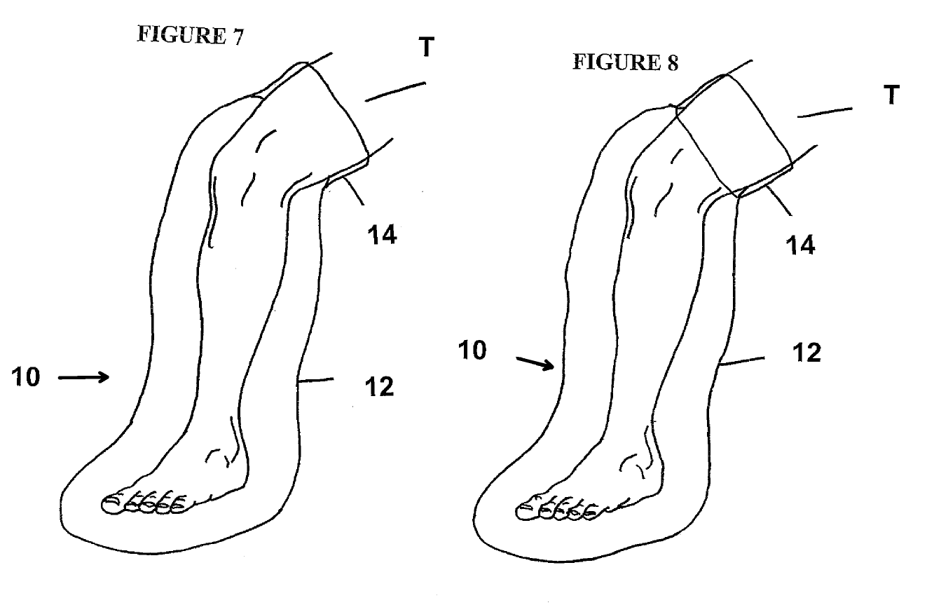

Referring now to FIGS. 7 and 8, again where like reference numerals are used to denote similar or like parts to that of FIGS. 5 and 6, the sleeve portion 12 is substantially elongate, to fit both the foot, lower leg, knee and lower thigh region of a user therein. In addition, the tapered cuff portion is typically sized with a greater average diameter to fit around the thigh T of a user.

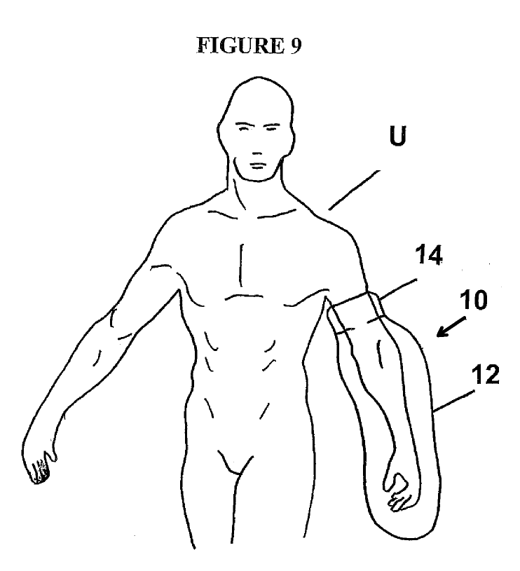

Referring now to FIG. 9, where like reference numerals are used to denote similar or like parts, a differently shaped and elongated sleeve portion 12 is shown which is adapted for enclosing the hand and forearm of a user U.

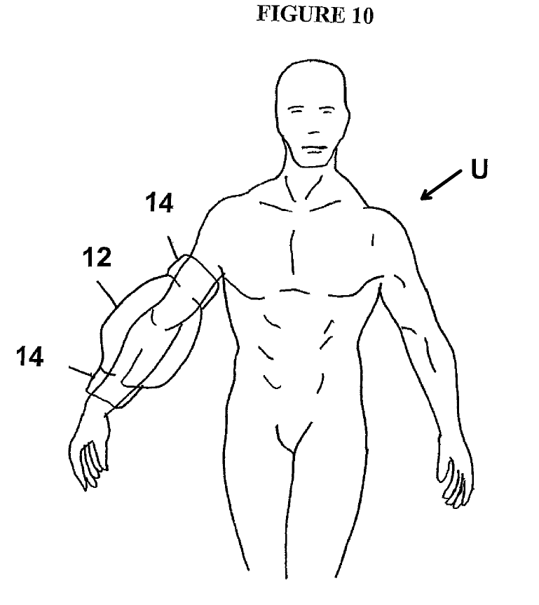

Referring now to FIG. 10, where like reference numerals are used to denote similar or like parts, a further modified sleeve portion 12 includes a pair of opposing cuff portions 12. The upper cuff portion seals against the user's upper arm and the lower cuff portion seals against the forearm. The sleeve 12 this isolates the user's elbow region, whilst still providing for hand and arm mobility. A similar arrangement can be adapted for positioning around a user's knee region.

The embodiments described above most typically have medical applications, for protecting cuts, abrasions, burns, plasters, bandages etc. against moisture, gas, contact, knocking and bumping. However, the arrangements can also be used in work and domestic applications wherever similar limb protection is required. The embodiments are particularly adapted for user by a user in showering and bathing applications.

Whilst the invention has been described with reference to a number of preferred embodiments, it should be appreciated that the invention can be embodied in many other forms.

What is claimed is:

1. A limb protection device for protecting at least one section of a user's limb, the device comprising:
a sleeve for use in enclosing the at least one limb section, the sleeve being sealed at one end; and
at least one flexible cuff integral with the sleeve and away from which the sleeve extends, the cuff being sealed at one end, and having a tapered profile arranged to allow an appropriately sized opening to be cut into the device, whereby the appropriately sized opening in the cuff is selectable to stretch around and compress against that part of the user's limb extending therethrough, to prevent the ingress of matter into or egress of matter out of the device immediately at the device opening, and wherein the compressive force exerted against that part of the user's limb prevents damage to the user's limb.

2. The device as recited in claim 1, wherein the cuff further comprises a series of spaced-apart protrusions extending along the length of the cuff, wherein the protrusions provide structural integrity to the cuff.

3. The device as recited in claim 2, wherein the protrusions are integrally moulded into the cuff.

4. The device as recited in claim 2, wherein the protrusions are of a substantially semi-circular shape.

5. The device as recited in claim 1, wherein the cuff has a width that is greater than a wall thickness of the sleeve.

6. The device as recited in claim 1, wherein the cuff is a continuation of the sleeve and is formed from at least one of the same material as the sleeve, a different material from the sleeve, and a more dense form of the same material as the sleeve.

7. The device as recited in claim 1, wherein the cuff is formed from a resilient polymeric material such as latex, or elastic impregnated plastic, to enable its stretching during fitting and removal of the device to a limb.

8. The device as recited in claim 7, wherein the sleeve comprises a releasably openable opening at the sealed end for enabling access to the limb section when the device is fitted to a user.

9. The device as recited in claim 8, wherein a press-seal opening is provided at the sealed end of the sleeve.

10. The device as recited in claim 7, wherein the sleeve is adapted for receiving a user's hand or foot therein or the sleeve is adapted for receiving the limb right therethrough, with two cuffs being located on opposing sides of the limb section that is enclosed within the device.

11. The device as recited in claim 7, wherein at least one of the sleeve and the cuff is transparent.

12. The device as recited in claim 7, wherein the sleeve is formed from a polymer that is integrally moulded with the cuff.

13. The device as recited in claim 7, wherein a therapeutic agent is provided in an interior of the device.

14. The device as recited in claim 1, further including a plurality of weakened zones wherein the weakened zone is hand manipulable to allow a user to effect a cut and thereby select an appropriately sized opening.

15. The device as recited in claim 1 wherein at least one of the cuff and sleeve is coloured.

16. The device as recited in claim 15, wherein the colour of the cuff or sleeve serves as an indicator of at least one of the size, sterility or therapeutic agent contained within the device.

17. The device as recited in claim 1 wherein the device is manufactured in a sterile environment and is packaged such that the device remains sterile until used by a user.

18. The device as recited in claim 1, wherein there are provided two flexible cuffs, each flexible cuff including a marker to allow a user to differentiate between each of the two cuffs.

19. The device as recited in claim 18 wherein the marker is a colouring of the cuff.

20. A method for applying a limb protection device to a user, comprising the steps of appropriately sizing a cuff of the limb device to ensure that the cuff is capable of stretching around and compressing against the user's limb, and applying the device to the user's limb such that the user's limb extends therethrough, to protect the ingress of matter into or the egress of matter out of the device immediately at the device opening.
